(19) United States
(12) Patent Application Publication
(10) Pub. No.: US 2026/0026693 A1
(43) Pub. Date: Jan. 29, 2026

(54) FLEXIBLE SENSING MODULE

(71) Applicant: Advanced Semiconductor Engineering, Inc., Kaohsiung (TW)

(72) Inventors: Yu-Chih LEE, Kaohsiung (TW); Ming-Ching LIN, Kaohsiung (TW); Kai-Lun YU, Kaohsiung (TW)

(73) Assignee: Advanced Semiconductor Engineering, Inc., Kaohsiung (TW)

(21) Appl. No.: 18/786,422

(22) Filed: Jul. 26, 2024

(51) Int. Cl. A61B 5/00

(52) U.S. Cl. CPC A61B 5/0077; A61B 5/6886; A61B 5/7225; A61B 2560/0223; A61B 2562/164

(57) ABSTRACT

The present disclosure relates to a sensing module and a flexible sensing module. The flexible sensing module includes a first sensing element configured to detect a bio-signal of a surface of a user; a second sensing element configured to detect a deformation of the flexible sensing module and to generate a first signal; and a processing element configured to calibrate the bio-signal in response to the first signal.

### BACKGROUND

### 1. Technical Field

The present disclosure generally relates to a sensing module, and more particularly to a flexible sensing module, including an optical sensor detecting bio-signals and a deformation sensor detecting deformation.

### 2. Description of the Related Art

Demands for advanced technology healthcare have led to increased deployment of sensors to detect or otherwise receive health-related information (e.g., bio-signals) to monitor users' health status. Nevertheless, sensors integrated into flexible substrates for better contact with users' skin can be compromised by irregularities in contact surfaces leading to distortion or movement at the sensing site, whereby accuracy of the sensor is impaired. Therefore, an improved sensor module is needed.

### SUMMARY

In some embodiments, a flexible sensing module includes a first sensing element configured to detect a bio-signal of a surface of a user; a second sensing element configured to detect a deformation of the flexible sensing module and to generate a first signal; and a processing element configured to calibrate the bio-signal in response to the first signal.

In some embodiments, a sensing module includes a flexible substrate; a first sensing element disposed on the flexible substrate and configured to detect a bio-signal; a second sensing element vertically overlapping the first sensing element and configured to detect a deformation of the sensing module to generate a first signal; and a processing element configured to calibrate the bio-signal in response to the first signal.

In some embodiments, a flexible sensing module includes a first sensing element configured to detect a bio-signal of a target of a user; and a processing element configured to calibrate the bio-signal in response to a distance variation between the first sensing element and the target.

### BRIEF DESCRIPTION OF THE DRAWINGS

Aspects of the present disclosure are readily understood from the following detailed description when read with the accompanying figures. It should be noted that various features may not be drawn to scale. The dimensions of the various features may be arbitrarily increased or reduced for clarity of discussion.

FIG. 1 is a cross-section of a sensing module, in accordance with some embodiments of the present disclosure.

FIG. 1A is a functional block diagram of the sensing module of FIG. 1, in accordance with some embodiments of the present disclosure.

FIG. 2 is a cross-section of a sensing module, in accordance with some embodiments of the present disclosure.

FIG. 3 is a cross-section of a sensing module, in accordance with some embodiments of the present disclosure.

FIG. 4 is a cross-section of a sensing module, in accordance with some embodiments of the present disclosure.

FIG. 5 is a graph illustrating time versus voltage of a deformation sensor, in accordance with some embodiments of the present disclosure.

FIG. 6 is a graph illustrating applied force versus resistance of a deformation sensor, in accordance with some embodiments of the present disclosure.

FIGS. 7A, 7B, and 7C illustrate a sensing module contacting a tissue phantom, in accordance with some embodiments of the present disclosure.

Common reference numerals are used throughout the drawings and the detailed description to indicate the same or similar elements. The present disclosure will be more apparent from the following detailed description taken in conjunction with the accompanying drawings.

### DETAILED DESCRIPTION

The following disclosure provides different embodiments, or examples, for implementing different features of the provided subject matter. Specific examples of components and embodiments are recited herein. These are, of course, merely examples and are not intended to be limiting. In the present disclosure, reference to the formation of a first feature over or on a second feature in the description that follows may include embodiments in which the first and second features are formed in direct contact, and may also include embodiments in which additional features may be formed between the first and second features, such that the first and second features may not be in direct contact. The present disclosure may repeat reference numerals and/or letters in the various examples. This repetition is for simplicity and clarity and does not in itself dictate a relationship between the various embodiments and/or configurations discussed.

Embodiments of the present disclosure are discussed in detail as follows. It should be appreciated, however, that the present disclosure provides many applicable concepts that can be embodied in a wide variety of specific contexts. The specific embodiments discussed are merely illustrative and do not limit the scope of the disclosure. Detection accuracy in a flexible sensing module with optical sensor for bio-signal and deformation sensor for deformation of the optical sensor can be improved when the flexible sensing module contacts a surface with a particular curvature or profile and the received bio-signal is calibrated based on the deformation caused by the curvature or elongation, so that the accuracy of the flexible sensing module can be improved.

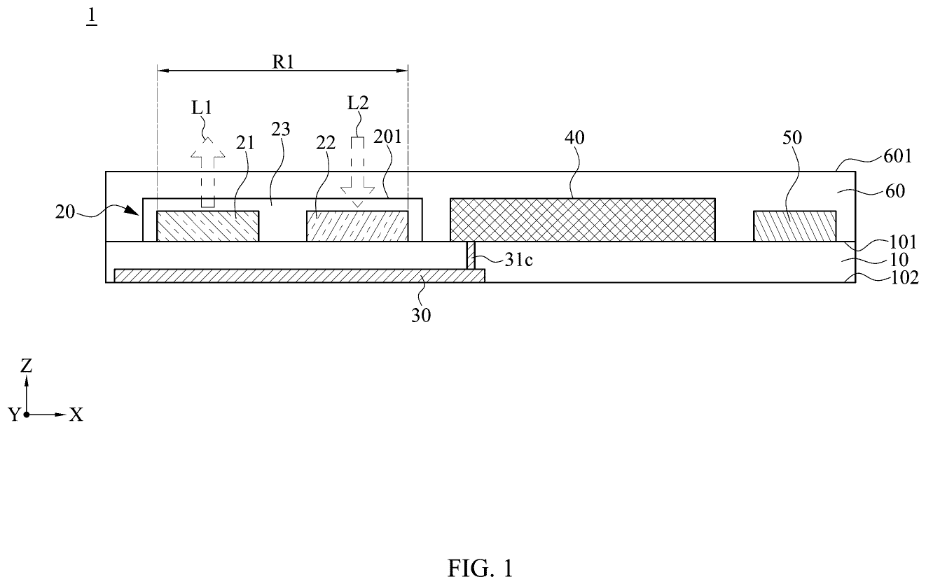

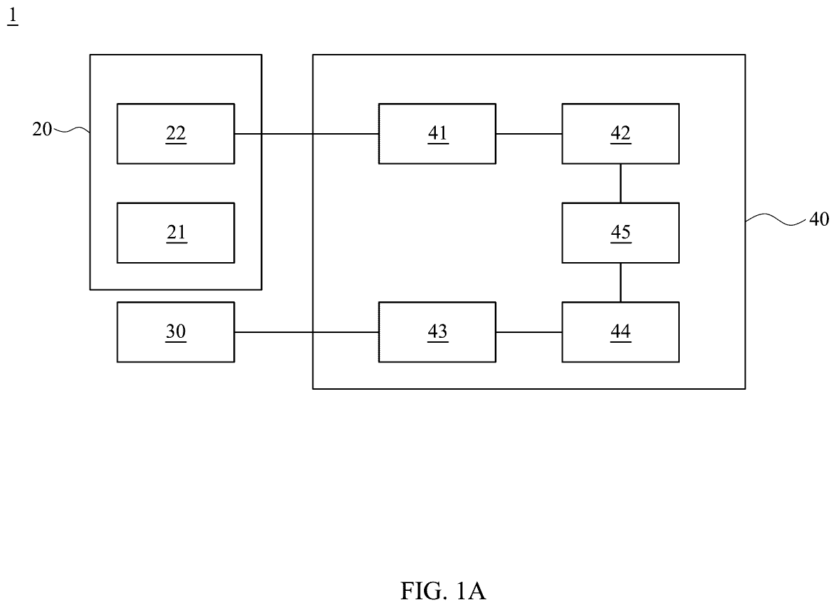

FIG. 1 is a cross-section of a sensing module 1, in accordance with some embodiments of the present disclosure. In some embodiments, the sensing module 1 may be utilized in a healthcare chip. The sensing module 1 can be utilized in a wearable electronic device, such as a smart watch, bracelet, healthcare patch, or other sensor for detecting bio-signals of the user. Referring to FIG. 1, the sensing module 1 may include a substrate 10, two sensing elements 20 and 30, a processing unit 40, a battery 50, and an encapsulant 60.

The substrate 10 may have a top surface 101 and a bottom surface 102 opposite to the top surface 101. The substrate 10 may be a flexible substrate. For example, the substrate 10 may be a flexible circuit board (FCB). In some embodiments, the substrate 10 may be transparent. In another embodiment, the substrate 10 may be opaque. In some embodiments, the shape of the substrate 10 may flexibly adjusted, such that the sensing module 1 can cover and be conformal to a detection site when attached thereto. For example, the shape, material, or the outline of the substrate 10 can be adjustable (e.g., twistable, stretchable, expandable, bendable, or flexible) to fit the contact shape of the user. In some embodiments, when the sensing module 1 is attached to the user, the top surface 101 of the substrate 10 may face the contact portion of the user.

The sensing element 20 may be disposed on the top surface 101 of the substrate 10. The sensing element 20 may be configured to detect a bio-signal of a surface of a user. In some embodiments, the sensing module 1 may contact and adhere to the user's skin. The sensing element 20 may be close to the user's skin so as to detect the bio-signal. In some embodiments, the bio-signals may be indicative of blood oxygen, heartbeat, haem, bilirubin, collagen, a body composition, or a body water.

In some embodiments, the bio-signals may be detected from a user when the sensing module 1 is worn by the user, and the sensing element 20 may be configured to be located between a portion of the user (not shown) and the substrate 10. In some embodiments, the sensing element 20 may be located at a first region R1 of the substrate 10. The first region R1 may be referred to as a sensing region of the sensing module 1. That is, when the sensing module 1 is attached on or worn by a user, a bio-signal of the user can be detected at the first region R1 by the sensing element 20. In some embodiments, the sensing element 20 may have a top surface 201 opposite to the substrate 10 and facing the user (not shown) when the sensing module 1 is attached on the user.

In some embodiments, the sensing element 20 may include an optical sensor. The optical sensor may be configured to detect blood oxygen, heartbeat, haem, bilirubin, collagen, a body composition, or a body water. The sensing element 20 may be an optical transceiver. In some embodiments, the sensing element 20 may include a light emitter 21 and an optical receiver 22. The light emitter 21 can be configured to emit light at different wavelengths. For example, the light emitter 21 may be configured to emit visible light, infrared light, ultraviolet light, or any other suitable light. In some embodiments, the light emitter 21 may include a LED.

The optical receiver 22 may be configured to receive reflected light from an object to be detected. For example, the optical receiver 22 may include a photodiode. In some embodiments, the light emitter 21 may be configured to emit a light L1 at a suitable wavelength to an object (for example, a skin, a finger, or an arm of the user). The light L1 is reflected by a target (inside the body of the user) to become reflected light L2. The optical receiver 22 can be configured to receive reflected light L2. In some embodiments, an intensity difference between the light L1 and reflected light L2 can be used to determine tissue composition, so as to obtain the bio-signals of the user.

In some embodiments, the intensity difference may depend on a distance (i.e., the light path length) that the light L1 and L2 go through. In some embodiments, the sensing element 20 may determine the tissue compositions according to the Beer-Lambert law.

In some embodiments, the sensing element 20 may further include a protective material 23 covering the light emitter 21 and the optical receiver 22. The protective material 23 may encapsulate the light emitter 21 and the optical receiver 22. The protective material 23 may be filled between the light emitter 21 and the optical receiver 22. In some embodiments, the protective material 23 may be transparent or can allow light within specific wavelength to pass.

In some embodiments, the sensing element 30 may be disposed within the substrate 10. The sensing element 30 may be embedded within the substrate 10. The sensing element 30 may be disposed below the sensing element 20. The sensing element 30 may overlap the sensing element 20 vertically. The sensing element 30 does not overlap the first sensing element horizontally. In some embodiments, the sensing element 30 may be exposed by the substrate 10. The bottom surface of the sensing element 30 may be exposed by the bottom surface 102 of the substrate 10. In other embodiments, the bottom surface of the sensing element 30 may protrude from the bottom surface 102 of the substrate 10. In some embodiments, a size of the sensing element 30 may be greater than or substantially identical to a size of the sensing element 20. For example, the sensing element 30 may be wider than or substantially identical to the sensing element 20. From the top view, the area of the sensing element 30 may be greater than or substantially identical to that of the sensing element 20.

In some embodiments, the sensing element 30 may be configured to detect a deformation of the sensing element 30 and generate a signal indicative of the deformation. In some embodiments, the sensing element 30 may be configured to detect a deformation at the first region R1 and to generate a signal corresponding to the deformation. The sensing element 30 may be located at the first region R1 of the substrate 10 and overlap the sensing element 20 vertically. In some embodiments, the sensing element 30 may include a deformation sensing film. In some embodiments, the sensing element 30 may be a resistive sensing thin film. For example, the sensing element 30 may include a piezoelectric film. In some embodiments, the sensing element 30 may include a variable resistance as a function of a deformation amount. The stress dependent voltage drop of the sensing element 30 may be calculated according to formula Eq. 1, as follows.

Vᵣ=R₀I[1+π_{L}σₓₓ+π_{T}(σ_{yy}+σ_{zz})] [Eq. 1], in which R₀ denotes a stress free resistance; I denotes an applied current; π_{L} denotes a transverse piezoresistive coefficient; π_{T} denotes a longitudinal piezoresistive coefficient; and σₓₓ, σ_{yy}, σ_{zz} denote a tensile stress component along x, y, and z axes, respectively.

In some embodiments, the processing unit 40 may be disposed on the top surface 101 of the substrate 10. The processing unit 40 may be disposed adjacent to the sensing element 20. The processing unit 40 may overlap the sensing element 20 horizontally. The processing unit 40 may be electrically connected to the sensing element 20, such that the bio-signals can be transmitted to the processing unit 40 for subsequent processing. For example, the processing unit 40 may filter, amplify, and/or digitize the bio-signals. In some embodiments, the processing unit 40 may be electrically connected to the sensing element 20 through one or more conductive elements, such as redistribution layers, conductive traces, conductive vias, or the like (not shown).

In some embodiments, the processing unit 40 may be disposed on the sensing element 30. The processing unit 40 may partially overlap the sensing element 30 vertically. The processing unit 40 may be electrically connected to the sensing element 30 through a conductive structure 31c, such that the signals obtained by the sensing element 30 can be transmitted to the processing unit 40 for subsequent processing. For example, the processing unit 40 may filter, amplify, and/or digitize the signals detected by the sensing element 30. In some embodiments, the processing unit 40 may be configured to calibrate the bio-signals detected by the sensing element 20 in response to the signals detected by the sensing element 30.

The processing unit 40 may include a processing element, one or more amplifiers, and one or more analog-to-digital converters (ADC). Details of the processing unit 40 are shown in FIG. 1A.

In some embodiments, the conductive structure 31c may be disposed between the sensing element 30 and the processing unit 40. The conductive structure 31c may electrically connect the sensing element 30 to the processing unit 40, such that the signals detected by the sensing element 30 can be transmitted to the processing unit 40. The conductive structure 31c may include a conductive via, a conductive pillar, a conductive trace, or a combination thereof. In some embodiments, the conductive structure 31c may include metals or alloys.

In some embodiments, the battery 50 may be disposed on the top surface 101 of the substrate 10. The battery 50 may be disposed adjacent to the processing unit 40. The battery 50 may overlap the sensing element 20 horizontally. In some embodiments, battery 50 may be spaced apart from the sensing element 20, the sensing element 30, and the processing unit 40. The battery 50 may be electrically connected to the sensing element 20, the sensing element 30, and the processing unit 40. In other embodiments, the battery 50 may be located at any suitable locations. In some embodiments, the battery 50 may be rigid, which means it is unbendable, non-flexible. Therefore, the battery 50 may be spaced apart from the sensing area so that the deformation can be detected accurately.

In some embodiments, the encapsulant 60 may be disposed on the top surface 101 of the substrate 10. The encapsulant 60 may cover or encapsulate the sensing element 20, the processing unit 40, and the battery 50. The top surface 101 of the substrate 10 may be entirely covered by the encapsulant 60. The encapsulant 60 may be disposed between the sensing element 20 and the processing unit 40. The encapsulant 60 may be disposed between the processing unit 40 and the battery 50. A lateral surface of the encapsulant 60 may be substantially coplanar with a lateral surface of the substrate 10. In some embodiments, the encapsulant 60 may have a top surface 601, which can contact the user when the sensing module 1 is worn by or attached to the user. In some embodiments, the sensing element 20 may be configured to emit optical signals toward the top surface 601 (to the user's body) so as to detect or sense the bio-signal through the reflected optical signals.

In some embodiments, the encapsulant 60 may be transparent. In some arrangements, the encapsulant 60 may include an epoxy resin, a molding compound (e.g., an epoxy molding compound or another molding compound), a material with a silicone dispersed therein, or a combination thereof.

In some embodiments, the sensing element 20 may be configured to detect a bio-signal of a target of a user (not shown). When the sensing module 1 is worn by the user, the sensing element 20 (for example, an optical sensor) may obtain the bio-signal based on the distance between the sensing element 20 and the target of the user. For example, the sensing element 20 may determine the distance (i.e., optical path) between the sensing element 20 and the target of the user based on the intensity difference of the light. In some embodiments, the target of the user may be a specific tissue of the user.

Because the sensing module 1 may be elongated or bent when it is worn by the user, the distance determined by the sensing element 20 may be inaccurate. Therefore, the sensing element 30 may be arranged adjacent to the sensing element 20 and configured to determine a distance variation between the sensing element 20 and the target of the user. The distance variation may be caused by a bending curvature of the sensing module 1 when the sensing module 1 is worn by the user. In some embodiments, the distance variation may be determined based on a deformation of the sensing element 30 (or a force applied thereon). In some embodiments, the processing unit 40 may be configured to calibrate the bio-signal in response to the distance variation between the sensing element 20 and the target of the user. In some embodiments, the processing unit 40 may include a memory (not shown) storing a database of correction parameters that is obtained by testing the sensing module 1 with tissue phantoms before use. The processing unit 40 may be configured to calibrate the bio-signal in response to the distance variation between the sensing element 20 and the target of the user based on the database of correction parameters, such that the variations resulted from the contacting surface of the sensing module 1 and the user can be calibrated. Accordingly, the accuracy of the sensing module 1 can be improved.

FIG. 1A is a functional block diagram of a sensing module 1 of FIG. 1, in accordance with some embodiments of the present disclosure. Referring to FIG. 1A, the processing unit 40 includes two amplifiers 41 and 43, two analog-to-digital converters (ADC) 42 and 44, and a processing element 45. In some embodiments, the processing unit 40 may include a memory (not shown) storing a database of the correction parameters that is obtained by testing the sensing module 1 with tissue phantoms before use. In some embodiments, the processing element 45 may be configured to process the calibration of the bio-signal detected by the sensing element 20 based on the signals detected by the sensing element 30 and the database of the correction parameters.

In some embodiments, the amplifier 41 may be electrically connected to the sensing element 20 and configured to amplify the bio-signal. The amplifier 41 may be electrically connected to the optical receiver 22 to receive the bio-signal. The amplifier 41 may be coupled between the optical receiver 22 and the ADC 42. In some embodiments, the amplifier 41 may be an operational amplifier. For example, the amplifier 41 may be a single-ended amplifier or a differential amplifier. The ADC 42 may be electrically connected to the amplifier 41 to receive the amplified bio-signal. The ADC 42 may be electrically connected between the amplifier 41 and the processing element 45. The ADC 42 may be configured to convert the bio-signal to digital, such that the processing element 45 can be configured to process the bio-signal.

In some embodiments, the amplifier 43 may be electrically connected to the sensing element 30 and configured to amplify the signal detected by the sensing element 30. In some embodiments, the amplifier 43 may be configured to amplify the signal associated with the deformation. The amplifier 43 may be coupled between the sensing element 30 and the ADC 44. In some embodiments, the amplifier 43 may be an operational amplifier. For example, the amplifier 43 may be a single-ended amplifier or a differential amplifier.

The ADC 44 may be electrically connected to the amplifier 43 to receive the amplified signal. The ADC 44 may be electrically coupled between the amplifier 43 and the processing element 45. The ADC 44 may be configured to convert the signal to digital, such that the processing element 45 can be configured to process the signal.

The processing element 45 may be configured to receive the bio-signal detected by the sensing element 20 and the signal detected by the sensing element 30 associated with deformation. Accordingly, the processing element 45 may be configured to calibrate the bio-signal in response to the signal associated with deformation. In some embodiments, the processing element 45 may include a processor or a controller, for example, a central processing unit (CPU), a microprocessor unit (MPU), a microcontroller unit (MCU), an application-specific integrated circuit (ASIC), or another type of processing integrated circuit.

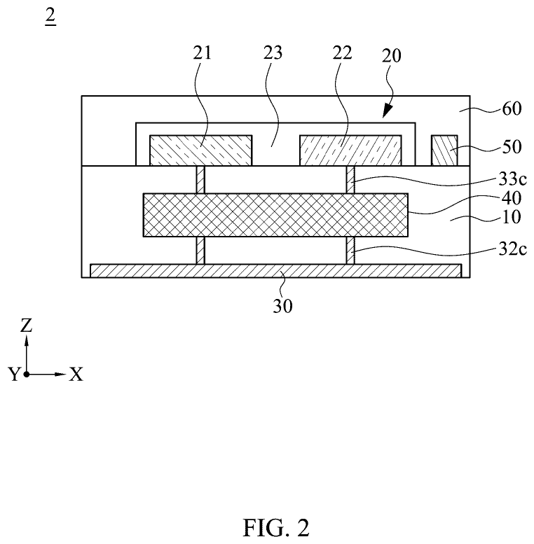

FIG. 2 is a cross-section of a sensing module 2, in accordance with some embodiments of the present disclosure. The sensing module 2 of FIG. 2 is similar to the sensing module 1 of FIG. 1, but with a different arrangement. In some embodiments, the processing unit 40 may be embedded within the substrate 10. The processing unit 40 may be disposed under the sensing element 20. The processing unit 40 may be disposed between the sensing element 20 and the sensing element 30. The processing unit 40 may overlap the sensing element 20 and the sensing element 30 vertically. In some embodiments, the encapsulant 60 may cover or encapsulate the sensing element 20 and the battery 50.

In some embodiments, the sensing element 20 may be positioned at a center of the sensing module 2. That is, the sensing element 20 may be located at a center of the substrate 10. In some embodiments, the sensing element 30 may be positioned at a center of the sensing module 2. That is, the sensing element 30 may be located at a center of the substrate 10. The sensing element 30 may be disposed below the sensing element 20. The sensing element 30 may overlap the sensing element 20 vertically. In some embodiments, a projection of the sensing element 20 on the substrate 10 may be completely within a projection of the sensing element 30 on the substrate 10.

In some embodiments, the sensing module 2 includes one or more conductive structures 32c and 33c. The conductive structures 32c may electrically connect the processing unit 40 and the sensing element 30. The conductive structures 33c may electrically connect the processing unit 40 and the sensing element 20. The conductive structures 32c and 33c are similar to the conductive structure 31c in FIG. 1, and thus details thereof are omitted for clarity.

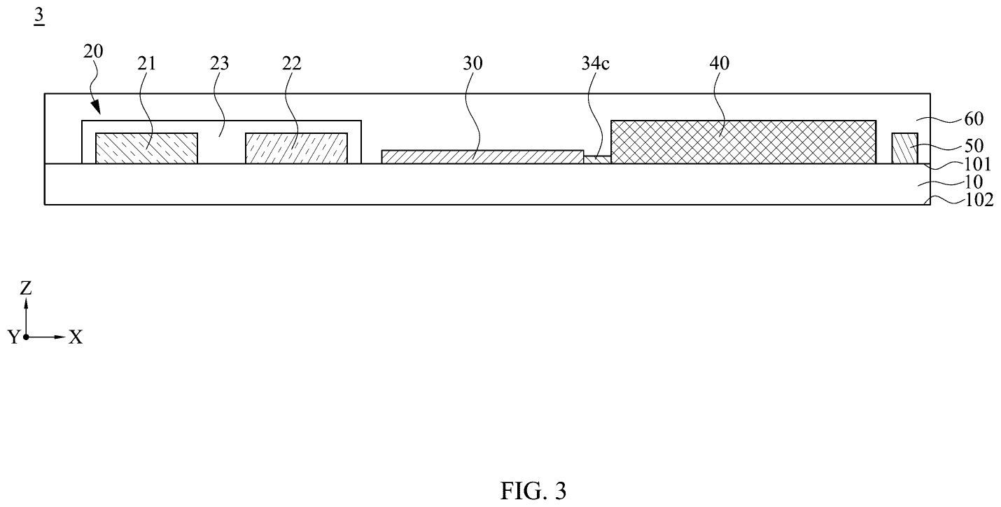

FIG. 3 is a cross-section of a sensing module 3, in accordance with some embodiments of the present disclosure. The sensing module 3 of FIG. 3 is similar to the sensing module 1 of FIG. 1, but with a different arrangement. In some embodiments, the sensing element 20, the sensing element 30, and the processing unit 40 may be disposed on the top surface 101 of the substrate 10.

In some embodiments, the sensing element 30 may be positioned at a center of the sensing module 3. That is, the sensing element 30 may be located at a center of the top surface 101 of the substrate 10. In some embodiments, the sensing element 30 may be disposed adjacent to the sensing element 20. The sensing element 30 may overlap the sensing element 20 horizontally. The sensing element 20 may be disposed at a side of the substrate 10, and the processing unit 40 may be disposed at an opposite side. In some embodiments, the sensing element 30 may be disposed between the sensing element 20 and the processing unit 40. In other embodiments, the sensing element 30 may surround the sensing element 20.

In some embodiments, the sensing module 3 includes one or more conductive structures 34c. The conductive structures 34c may electrically connect the processing unit 40 and the sensing element 30. The conductive structures 34c are similar to the conductive structure 31c in FIG. 1, and thus details thereof are omitted for clarity. For example, the conductive structures 34c may be conductive traces arranged in a specific pattern.

In some embodiments, the encapsulant 60 may cover or encapsulate the sensing element 20, the sensing element 30, the processing unit 40, and the battery 50. In some embodiments, the encapsulant 60 may be disposed between the sensing elements 20 and 30. In some embodiments, the encapsulant 60 may be disposed between the sensing element 30 and the processing unit 40. The encapsulant 60 may cover or encapsulate the conductive structures 34c.

The battery 50 may be rigid, unbendable, non-flexible, and thus the battery 50 may be spaced apart from the sensing area of the sensing element 30, such that the battery 50 would not affect the accuracy of the sensing element 30.

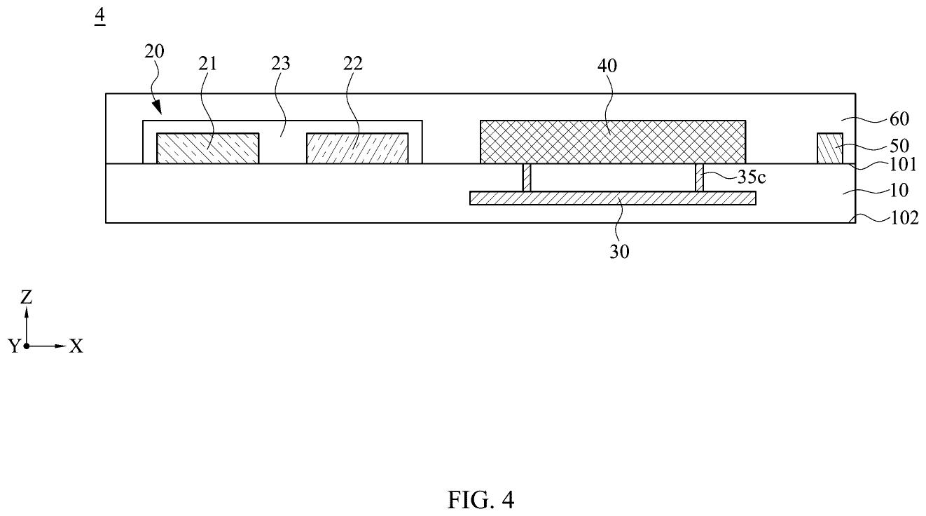

FIG. 4 is a cross-section of a sensing module 4, in accordance with some embodiments of the present disclosure. The sensing module 4 of FIG. 4 is similar to the sensing module 1 of FIG. 1, but with a different arrangement. In some embodiments, the sensing element 30 may be disposed under the processing unit 40 and embedded within the substrate 10. In some embodiments, the sensing element 30 may be completely covered by the substrate 10.

In some embodiments, the sensing element 30 may be disposed adjacent to the sensing element 20. The sensing element 30 may overlap the processing unit 40 vertically. The sensing element 20 may be disposed at a side of the substrate 10, and the processing unit 40 may be disposed at an opposite side.

In some embodiments, the sensing module 4 includes one or more conductive structures 35c. The conductive structures 35c may electrically connect the processing unit 40 and the sensing element 30. The conductive structures 34c are similar to the conductive structure 31c in FIG. 1, and thus details thereof are omitted for clarity. For example, the conductive structures 34c may include conductive pillars or vias.

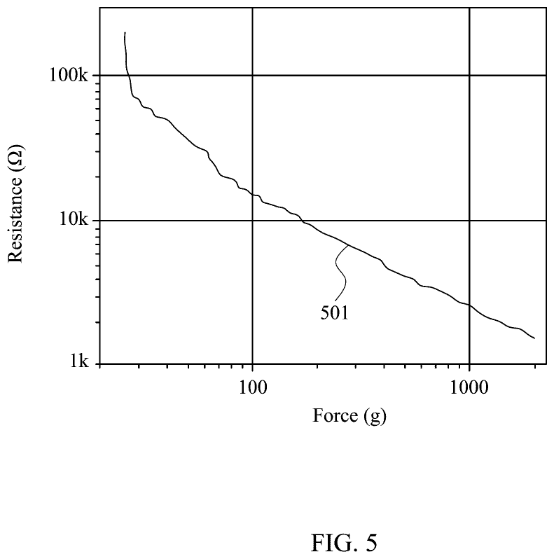

FIG. 5 is a graph illustrating applied force versus resistance of a deformation sensor, in accordance with some embodiments of the present disclosure. FIG. 5 includes a line 501 showing a variable resistance of the deformation sensor under different forces applied thereon. In some embodiments, the deformation sensor may be the sensing element 30. The deformation sensor may be a deformation sensing film, for example, a piezoelectric film.

In some embodiments, the line 501 indicates that the resistance of the deformation sensor is less with greater force. In some embodiments, increased force applied to the deformation sensor may increase deformation, and thus the deformation sensor can detect the deformation amount according to the variable resistance. In some embodiments, the deformation sensor may include a variable resistance as a function of a deformation amount.

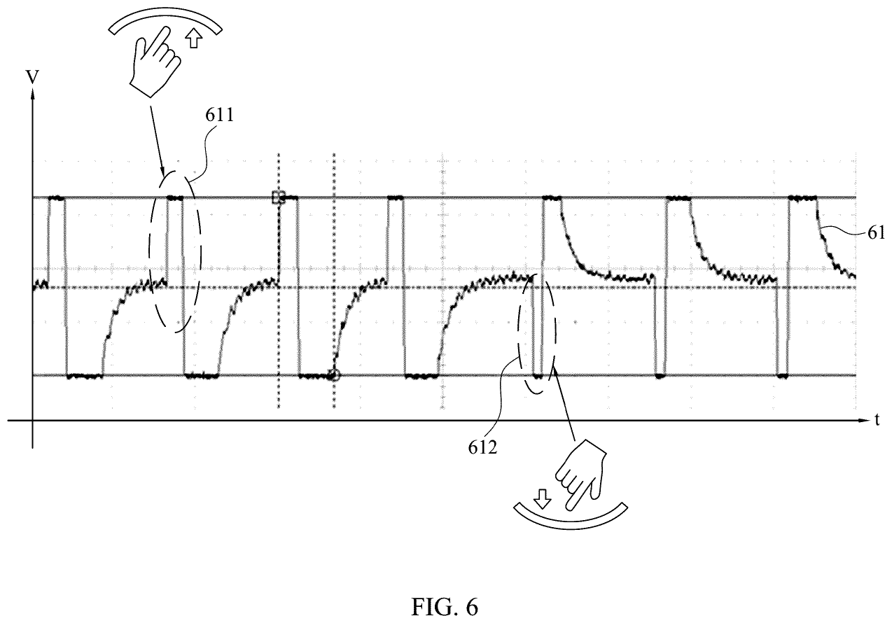

FIG. 6 is a graph illustrating time versus voltage of a deformation sensor, in accordance with some embodiments of the present disclosure. FIG. 6 includes a line 61 showing the detected voltage of the deformation sensor along the time line. The line 61 may include waveforms 611 and 612 indicative of operations applied to the deformation sensor.

In some embodiments, the deformation sensor may be the sensing element 30. The deformation sensor may be a deformation sensing film, for example, a piezoelectric film. When forces are applied to the deformation sensor at different strengths in different directions, the voltage line 61 may show corresponding waveforms. The waveform 611 may occur when upward force is applied to the deformation sensor. The waveform 612 may occur when downward force is applied to the deformation sensor.

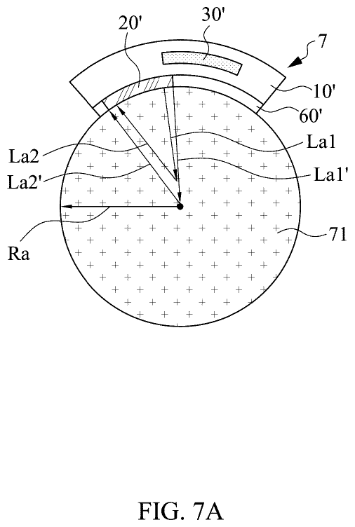

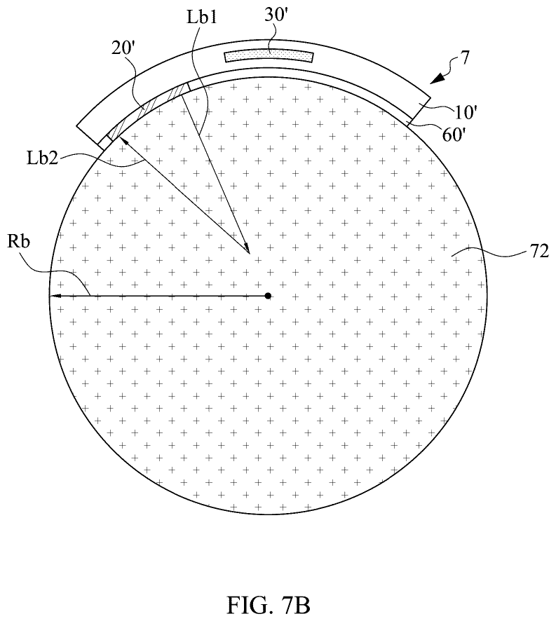

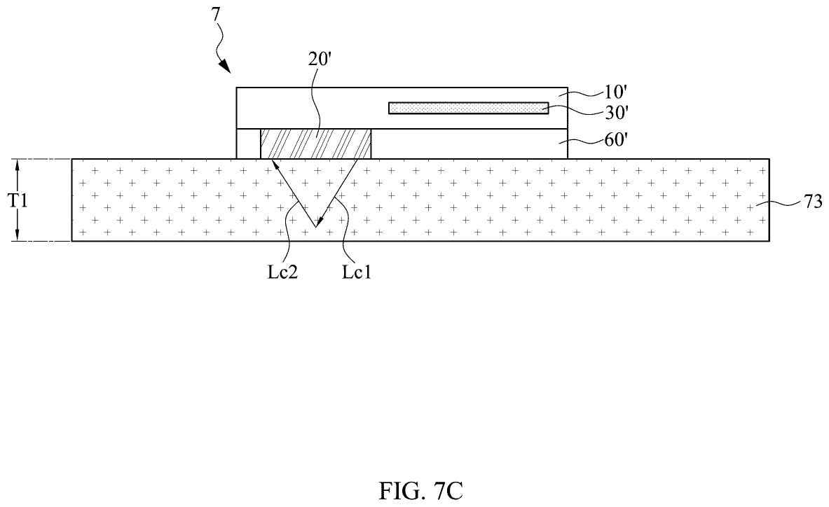

FIGS. 7A, 7B, and 7C illustrate a sensing module contacting a tissue phantom, in accordance with some embodiments of the present disclosure. In some embodiments, FIGS. 7A, 7B, and 7C show the sensing module contacting different tissue phantoms to collect correction parameters.

Referring to FIG. 7A, a sensing module 7 may be disposed on a tissue phantom 71. In some embodiments, the sensing module 7 may be adjustable (e.g., twistable, stretchable, expandable, bendable, or flexible) to conform to the contact shape of the tissue phantom 71. The tissue phantom 71 may be rounded. For example, the tissue phantom 71 may be a cylinder or a sphere. In some embodiments, the tissue phantom 71 may have a radius Ra. The curvature of the sensing module 7 may be substantially conformal to the contact curvature of the tissue phantom 71.

The sensing module 7 may include a substrate 10′, two sensing elements 20′ and 30′, and an encapsulant 60′. In some embodiments, the sensing module 7 may be one of sensing modules 1, 2, 3, or 4 of FIG. 1, 2, 3, or 4. Detailed descriptions of the sensing module 7 can be found in paragraphs associated with FIGS. 1, 2, 3, and 4, and thus are not repeated here.

In some embodiments, the sensing element 20′ may be configured to emit a light La1 toward the tissue phantom 71. The light La1 may be reflected and scattered in the tissue phantom 71. In some embodiments, the reflected light La2 (or scattered light) may be received by the sensing element 20′, such that the sensing element 20′ can determine the intensity difference of the light La1 and La2. In some embodiments, the sensing element 30′ may be configured to detect a deformation of the sensing module 7 caused by the bending curvature of the sensing module 7. As the radius Ra is already known, the signal associated with the deformation can be stored as correction parameters for use in actual operations.

In some embodiments, the sensing element 20′ may be configured to emit a light La1′ toward the tissue phantom 71. The light La1′ may have a wavelength different from the light La1. Comparing to the light La1, the light La1′ may be reflected and scattered by a deeper target within the tissue phantom 71. In some embodiments, the reflected light La2′ (or scattered light) may be received by the sensing element 20′, such that the sensing element 20′ can determine the intensity difference of the light La1′ and La2′. In some embodiments, the sensing element 30′ may be configured to detect a deformation of the sensing module 7 caused by the bending curvature of the sensing module 7. As the radius Ra is already known, the signal associated with the deformation can be stored as correction parameters for use in actual operations. Accordingly, although the sensing module 7 is bent with the same amount, the sensing element 20′ can detect targets at different locations/distances by using lights of different wavelengths.

In some embodiments, the sensing element 20′ may be configured to emit lights at different wavelengths. Different wavelengths may be suitable to detect the deep target (such as blood vessels, blood capillaries, muscles) or the shallow target (such as skin tissues). Therefore, the sensing element 20′ may be configured to detect the target at different locations using the lights (such as La1 and La1′) at the different wavelengths.

Referring to FIG. 7B, the sensing module 7 may be disposed on a tissue phantom 72. In some embodiments, the sensing module 7 may be adjustable (e.g., twistable, stretchable, expandable, bendable, or flexible) to fit the contact shape of the tissue phantom 72. The tissue phantom 72 may be rounded. For example, the tissue phantom 72 may be a cylinder or a sphere. In some embodiments, the tissue phantom 72 may have a radius Rb, which may be greater than the radius Ra of the tissue phantom 71. The curvature of the sensing module 7 may be substantially conformal to the contact curvature of the tissue phantom 72.

In some embodiments, the sensing element 20′ may be configured to emit a light Lb1 toward the tissue phantom 72. The light Lb1 may be reflected and scattered in the tissue phantom 72. In some embodiments, the reflected light Lb2 (or scattered light) may be received by the sensing element 20′, such that the sensing element 20′ can determine the intensity difference of the light Lb1 and Lb2. In some embodiments, the sensing element 30′ may be configured to detect a deformation of the sensing module 7 caused by the bending curvature of the sensing module 7. As the radius Rb is already known, the signal associated with the deformation can be stored as the correction parameters to be used in actual operations.

The sensing module 7 may be placed on or contact tissue phantoms of different sizes to collect correction parameters before use. When the sensing module 7 is worn by or attached on the user, the bio-signals detected by the sensing element 20′ can be calibrated in response to the signals associated with the deformation caused by the contact shape based on the correction parameters.

Referring to FIG. 7C, the sensing module 7 may be disposed on a tissue phantom 73. In some embodiments, the sensing module 7 may be adjustable (e.g., twistable, stretchable, expandable, bendable, or flexible) to fit the contact shape of the tissue phantom 73. The tissue phantom 73 may be flat. For example, the tissue phantom 73 may be a cube. In some embodiments, the tissue phantom 73 may have a thickness T1. When the sensing module 7 is tightly attached to the tissue phantom 73, the sensing module 7 may be elongated horizontally.

In some embodiments, the sensing element 20′ may be configured to emit a light Lc1 toward the tissue phantom 73. The light Lc1 may be reflected and scattered in the tissue phantom 73. In some embodiments, the reflected light Lc2 (or scattered light) may be received by the sensing element 20′, such that the sensing element 20′ can determine the intensity difference of the light Lc1 and Lc2. In some embodiments, the sensing element 30′ may be configured to detect a deformation of the sensing module 7 caused by the elongation of the sensing module 7. Since the tissue phantom 73 is flat with a known thickness T1, the signal associated with the deformation can be stored as the correction parameters to be used in actual operations.

Correction parameters can be obtained by testing the sensing module 7 with different tissue phantoms before use. The sensing module 7 may be configured to calibrate (by the non-shown processing unit) the bio-signals detected by the sensing element 20′ in response to the signals associated with the deformation caused by the contact shape of the user. This calibration allows the correction parameters to be applied, ensuring that the bio-signals accurately reflect the user's physiological state. By calibrating the bio-signals in this way, the sensing module 7 can provide more accurate and reliable data for monitoring the user's health. This calibration process can prevent bio-signals distortion by variations in contact shape.

Correction parameters can be obtained by testing the sensing module 7 with different tissue phantoms before use. The sensing module 7 may be configured to calibrate (by the non-shown processing unit) the bio-signals detected by the sensing element 20′ in response to the signals associated with the deformation caused by the contact shape of the user. This calibration allows the correction parameters to be applied, ensuring that the bio-signals accurately reflect the user's physiological state. By calibrating the bio-signals in this way, the sensing module 7 can provide more accurate and reliable data for monitoring the user's health. This calibration process could avoid the bio-signals from being distorted by the contact shape of the user.

Spatial descriptions, such as “above,” “below,” “up,” “left,” “right,” “down,” “top,” “bottom,” “vertical,” “horizontal,” “side,” “higher,” “lower,” “upper,” “over,” “under,” and so forth, are indicated with respect to the orientation shown in the figures unless otherwise specified. It should be understood that the spatial descriptions used herein are for purposes of illustration only, and that practical implementations of the structures described herein can be spatially arranged in any orientation or manner, provided that the merits of embodiments of this disclosure are not deviated from by such an arrangement.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, a first numerical value can be deemed to be “substantially” the same or equal to a second numerical value if the first numerical value is within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” perpendicular can refer to a range of angular variation relative to 90° that is less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Two surfaces can be deemed to be coplanar or substantially coplanar if a displacement between the two surfaces is no greater than 5 μm, no greater than 2 μm, no greater than 1 μm, or no greater than 0.5 μm. A surface can be deemed to be substantially flat if a displacement between a highest point and a lowest point of the surface is no greater than 5 μm, no greater than 2 μm, no greater than 1 μm, or no greater than 0.5 μm.

As used herein, the singular terms “a,” “an,” and “the” may include plural referents unless the context clearly dictates otherwise.

As used herein, the terms “conductive,” “electrically conductive” and “electrical conductivity” refer to an ability to transport an electric current. Electrically conductive materials typically indicate those materials that exhibit little or no opposition to the flow of an electric current. One measure of electrical conductivity is Siemens per meter (S/m). Typically, an electrically conductive material is one having a conductivity greater than approximately 104 S/m, such as at least 105 S/m or at least 106 S/m. The electrical conductivity of a material can sometimes vary with temperature. Unless otherwise specified, the electrical conductivity of a material is measured at room temperature.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. A flexible sensing module, comprising:
a first sensing element configured to detect a bio-signal of a surface of a user;
a second sensing element configured to detect a deformation of the flexible sensing module and to generate a first signal; and
a processing element configured to calibrate the bio-signal in response to the first signal.

2. The flexible sensing module of claim 1, wherein the first sensing element is closer to the surface of the user than the second sensing element is.

3. The flexible sensing module of claim 1, wherein the first sensing element is an optical transceiver, comprising a light emitter and an optical receiver.

4. The flexible sensing module of claim 3, wherein the light emitter is configured to emit lights at different wavelengths.

5. The flexible sensing module of claim 4, wherein the first sensing element is configured to detect a target to be detected at different locations using the lights at the different wavelengths.

6. The flexible sensing module of claim 1, further comprising a first amplifier electrically connected to the second sensing element and configured to amplify the first signal.

7. The flexible sensing module of claim 6, further comprising a first analog-to-digital converter (ADC) electrically connected between the first amplifier and the processing element, and configured to convert the first signal to digital, such that the processing element is configured to process the first signal.

8. The flexible sensing module of claim 1, further comprising a second amplifier electrically connected to the first sensing element and configured to amplify the bio-signal.

9. The flexible sensing module of claim 8, further comprising a second ADC electrically connected between the second amplifier and the processing element, and configured to convert the bio-signal to digital, such that the processing element is configured to process the bio-signal.

10. A sensing module, comprising:
a flexible substrate;
a first sensing element disposed on the flexible substrate and configured to detect a bio-signal;
a second sensing element vertically overlapping the first sensing element and configured to detect a deformation of the sensing module to generate a first signal; and
a processing element configured to calibrate the bio-signal in response to the first signal.

11. The sensing module of claim 10, wherein the second sensing element is positioned at a center of the flexible substrate.

12. The sensing module of claim 10, wherein the second sensing element is disposed within the flexible substrate.

13. The sensing module of claim 10, wherein the second sensing element non-overlaps the first sensing element horizontally.

14. The sensing module of claim 10, wherein a size of the second sensing element is greater than or identical to a size of the first sensing element.

15. The sensing module of claim 10, wherein the processing element is disposed under the first sensing element.

16. The sensing module of claim 10, further comprising a battery disposed on the flexible substrate and outside of a vertical projection of the second sensing element on the flexible substrate.

17. A flexible sensing module, comprising:
a first sensing element configured to detect a bio-signal of a target of a user; and
a processing element configured to calibrate the bio-signal in response to a distance variation between the first sensing element and the target.

18. The flexible sensing module of claim 17, further comprising a second sensing element configured to determine the distance variation.

19. The flexible sensing module of claim 18, wherein the second sensing element includes a deformation sensing film.

20. The flexible sensing module of claim 19, wherein the deformation sensing film includes a variable resistance as a function of a deformation amount.
